(19) United States
(12) Patent Application Publication
(10) Pub. No.: US 2004/0110929 A1
(43) Pub. Date: Jun. 10, 2004

(54) TF binding compound

(76) Inventors: Soren E. Bjorn, Lyngby (DK); Else Marie Nicolaisen, Frederiksberg (DK); Thomas Dock Steenstrup, Gentofte (DK)

Correspondence Address: NOVO NORDISK PHARMACEUTICALS, INC, 100 COLLEGE ROAD WEST, PRINCETON, NY, 08540, US

(21) Appl. No.: 10/617,619

(22) Filed: Jul. 11, 2003

Related U.S. Application Data
(60) Provisional application No. 60/404,568, filed on Aug. 19, 2002.

(30) Foreign Application Priority Data
Jul. 12, 2002 (DK) PA 2002 01099

(51) Int. Cl. C07K 14/745

(52) U.S. Cl. 530/384

(57) ABSTRACT

The present invention relates to novel compounds that bind to and inhibit the activity of tissue factor (TF) and mediate a cytolytic immune response.

### CROSS-REFERENCE TO RELATED APPLICATIONS

This application claims priority under 35 U.S.C. 119 of Danish application no. PA 2002 01099 filed Jul. 12, 2002 and U.S. application No. 60/404,568 filed Aug. 19, 2002, the contents of which are fully incorporated herein by reference.

### FIELD OF THE INVENTION

The present invention relates to novel compounds which bind to and inhibit the activity of tissue factor (TF) and mediate a cytolytic immune response. The invention also relates to pharmaceutical compositions comprising the novel compounds as well as their use in the treatment of or prophylaxis of diseases or disorders related to pathophysiological TF activity including cancer, inflammation, atherosclerosis and ischemia/reperfusion.

### BACKGROUND OF THE INVENTION

Tissue Factor (TF) is a cellular transmembrane receptor for plasma coagulation factor VIIa and formation of TF/VIIa complexes on the cell surface triggers the coagulation cascade in vivo. The TF/VIIa complex efficiently activates coagulation factors IX and X. The resultant protease factor Xa (Xa), activates prothrombin to thrombin, which in turn converts fibrinogen into a fibrin matrix.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis and ischemia/reperfusion. Thus TF is now recognised as a target for therapeutic intervention in conditions associated with increased expression.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

In addition to its established role as an initiator of the coagulation process, TF was recently shown to function as a mediator of intracellular activities either by interactions of the cytoplasmic domain of TF with the cytoskeleton or by supporting the VIIa-protease dependent signaling. Such activities may be responsible, at least partly, for the implicated role of TF in tumor development, metastasis and angiogenesis. Cellular exposure of TF activity is advantageous in a crisis of vascular damage but may be fatal when exposure is sustained as it is in these various diseased states. Thus, it is critical to regulate the expression of TF activity in maintaining the health.

A patient's immune system has several components, some of which are useful for cell therapy. In particular, antibody-dependent cell-mediated cytotoxicity (ADCC) has an important role in the destruction of many target cells, including tumor cells, by macrophages. Opsonization of target cells with immunoglobulin G (IgG) enhances the removal of these materials from a host. The role of macrophages in the destruction of target cells by ADCC in the presence of specific antibodies has been well established. While the selectivity of macrophage targeting is based on antibody specificity, the lytic attack on the target cells is triggered by Fc receptor-mediated ADCC.

Another component of the immune system is the activation of the complement system (Byrn, R. A, et al., Nature 344, 667-670, 1990). The two pathways of complement activation (the classical and the alternative pathways) are both directed at a central step in complement activation, the cleavage of C3. A single terminal pathway is the formation of a membrane attack complex (MAC). The classical pathway is normally activated by antigen-antibody complexes, where certain antibodies are complement fixing (capable of binding to complement to cause activation of the classical pathway). Activation of the classical pathway can be initiated with binding of C1q, the first factor of complement cascade, to the Fc region of immunoglobulin. Then, a cascade of proteolytic events results in the activation of C5 convertase, which cleaves CS into C5b and C5a. The C5b then binds C6, C7, C8 to form a C5b-8 complex. Binding of C9 molecules to C5b-8 forms C5b-9 (the MAC), which inserts into lipid bilayers and forms transmembrane channels that permit bidirectional flow of ions and macromolecules. By this mechanism, complement causes lysis of the cells.

Inactivated FVII (FVIIai) is FVIIa modified in such a way that it is catalytically inactive. FVIIai is thus not able to catalyze the conversion of FX to FXa, but still able to bind to TF in competition with active endogenous FVIIa and thereby inhibit the TF activity.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. International patent application WO 01/21661 relates to bivalent inhibitor of FVII and FXa.

Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

There is still a need in the art for improved compounds, which efficiently inhibit pathophysiological TF activity at relatively low doses and which do not produce undesirable side effects. The present invention provides compounds that act specifically on pathophysiological TF activity and at the same time elicit a cytolytic immune response in a patient.

### SUMMARY OF THE INVENTION

The present invention relates to a immunoconjugates of native human FVIIa or procoagulant variants thereof. One aspect relates to immunoconjugates, wherein native human FVIIa or procoagulant variants thereof have been chemically inactivated. The inactivation of the FVIIa proteolytic activity may be obtained in vitro by covalent active site inhibitors e.g. chloromethyl ketones. The conjugate has very high affinity for TF due to the increased affinity of the chemically inactivated binding domain as compared to the binding of native FVII. The high affinity provides a more efficacious and safe treatment of a patient in need thereof. The conjugate may also have a higher affinity for TF due an avidity effect in dimers, trimers or other multimers with multiple TF binding sites.

In a first aspect, the present invention relates to a compound having the formula A-(LM)-C, wherein A is a FVIIa polypeptide; LM is an optional linker moiety; C comprises an immunostimulatory effector domain; and wherein the compound binds to TF.

In a second aspect, the present invention relates to a pharmaceutical composition comprising an amount of a compound having the formula A-(LM)-C, wherein A is a FVIIa polypeptide; LM is an optional linker moiety; C comprises an immunostimulatory effector domain; and wherein the compound binds to TF; and a pharmaceutically acceptable carrier or excipient.

In a third aspect, the present invention relates to a compound for use as a medicament having the formula A-(LM)-C, wherein A is a FVIIa polypeptide; LM is an optional linker moiety; C comprises an immunostimulatory effector domain; and wherein the compound binds to TF.

In a further aspect, the present invention relates to the use of a compound having the formula A-(LM)-C, wherein A is a FVIIa polypeptide; LM is an optional linker moiety; C comprises an immunostimulatory effector domain; and wherein the compound binds to TF; for the manufacture of a medicament for preventing or treating disease or disorder associated with pathophysiological TF activity.

In a further aspect, the present invention relates to a polynucleotide construct encoding a Factor VII polypeptide conjugated to (LM)-C, wherein LM is an optional linker moiety and C comprises an immunostimulatory effector domain. In one embodiment, the polynucleotide construct is a vector. In one embodiment, the polynucleotide construct encodes a polypeptide comprising the sequence independently selected from the group consisting of SEQ ID NO:6, SEQ ID NO:7, SEQ ID NO:8 and SEQ ID NO:11. In one embodiment, the polynucleotide construct encodes a polypeptide which has the sequence of SEQ ID NO:6. In one embodiment, the polynucleotide construct encodes a polypeptide which has the sequence of SEQ ID NO:11. In one embodiment, the polynucleotide construct comprises the sequence independently selected from the group consisting of SEQ ID NO:9, SEQ ID NO:10, SEQ ID NO:12, and SEQ ID NO:13. In one embodiment, the polynucleotide construct encodes a polypeptide which has the sequence of SEQ ID NO:9. In one embodiment, the polynucleotide construct encodes a polypeptide which has the sequence of SEQ ID NO:12. In one embodiment, the polynucleotide construct encodes a polypeptide which has the sequence of SEQ ID NO:10. In one embodiment, the polynucleotide construct encodes a polypeptide which has the sequence of SEQ ID NO:13.

In a further aspect, the present invention relates to a host cell comprising the polynucleotide construct encoding a Factor VII polypeptide conjugated to (LM)-C, wherein LM is an optional linker moiety and C comprises an immunostimulatory effector domain. In one embodiment, the host cell is a eukaryotic cell. In one embodiment, the host cell is of mammalian origin. In one embodiment, the host cell is selected from the group consisting of CHO cells, HEK cells, and BHK cells. In one embodiment of the invention, the host cell is a hybridoma cell. In one embodiment, the host cell is an isolated lymphoid cell. In a further embodiment, the cell is isolated from a mouse. In one embodiment, the hybridoma cell is obtained by fusion of an antibody-producing lymphoid cell with an immortal cell to provide an antibody-producing hybridoma cell.

In a further aspect, the present invention relates a method for preventing or treating disease or disorder associated with pathophysiological TF activity, the method comprising contacting a TF-expressing cell with a compound having the formula A-(LM)-C, wherein A is a FVIIa polypeptide; LM is an optional linker moiety; C comprises an immunostimulatory effector domain; and wherein the compound binds to TF.

### BRIEF DESCRIPTION OF THE DRAWINGS

FIG. 1 is a graphic illustration of plasmid vector pFVII-Fc according to example 1, SEQ ID NO:13.

FIG. 2 illustrates the amino acid sequence of human Factor VII (SEQ ID NO:1); the DNA sequences of primers represented by SEQ ID NOs:2-5, the amino acid sequences of a human Factor VII variant with an alternatively spliced propeptide conjugated to the Fc domain of IgG1 (SEQ ID NO:6); the amino acid sequence of the Fc domain of human IgG1 (SEQ ID NO:7); the amino acid sequence of human Factor VII conjugated to the Fc domain of IgG1, in which X refers to GLA residues (SEQ ID NO:8); the cDNA sequence encoding human Factor VII with an alternatively spliced propeptide conjugated to the Fc domain of IgG1 (SEQ ID NO:9); the DNA sequence of the vector comprising the cDNA sequence encoding human Factor VII with an alternatively spliced propeptide conjugated to the Fc domain of IgG1 (SEQ ID NO:10); the amino acid sequence of human Factor VII conjugated to the Fc domain of IgG1 (SEQ ID NO:11); the cDNA sequence encoding human Factor VII conjugated to the Fc domain of IgG1 (SEQ ID NO:12); and the DNA sequence of the vector comprising the cDNA sequence encoding human Factor VII conjugated to the Fc domain of IgG1 (SEQ ID NO:13).

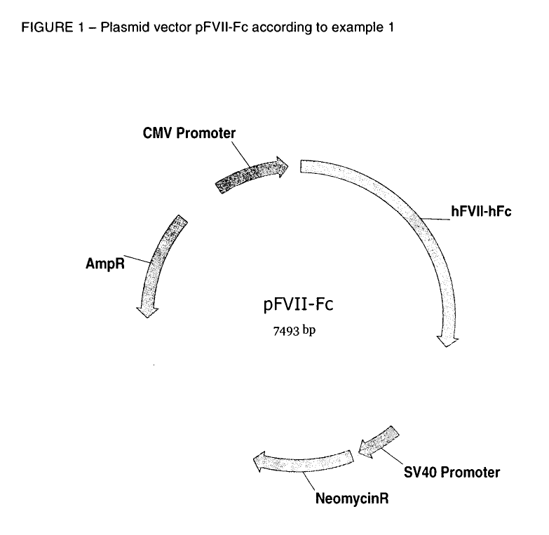

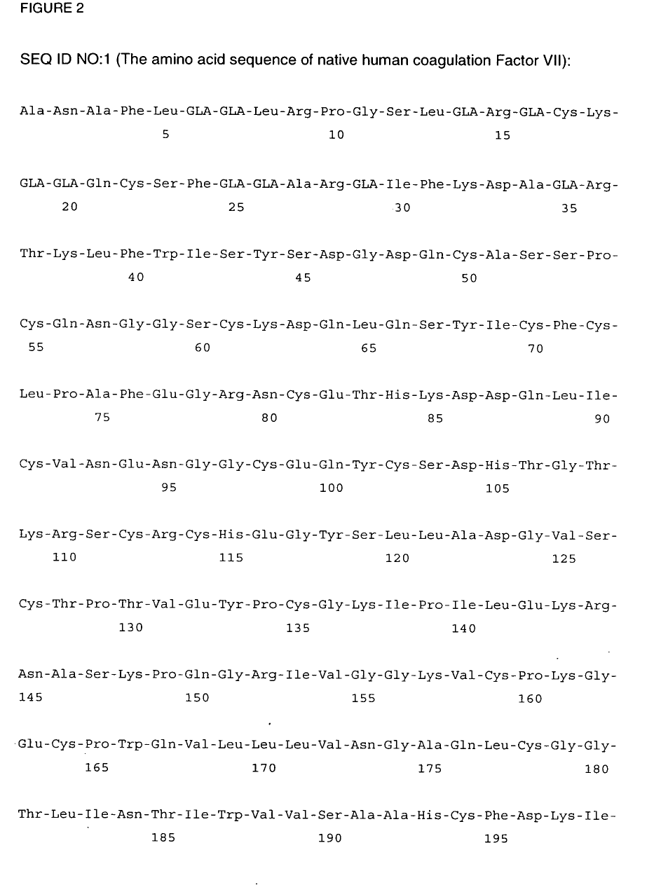

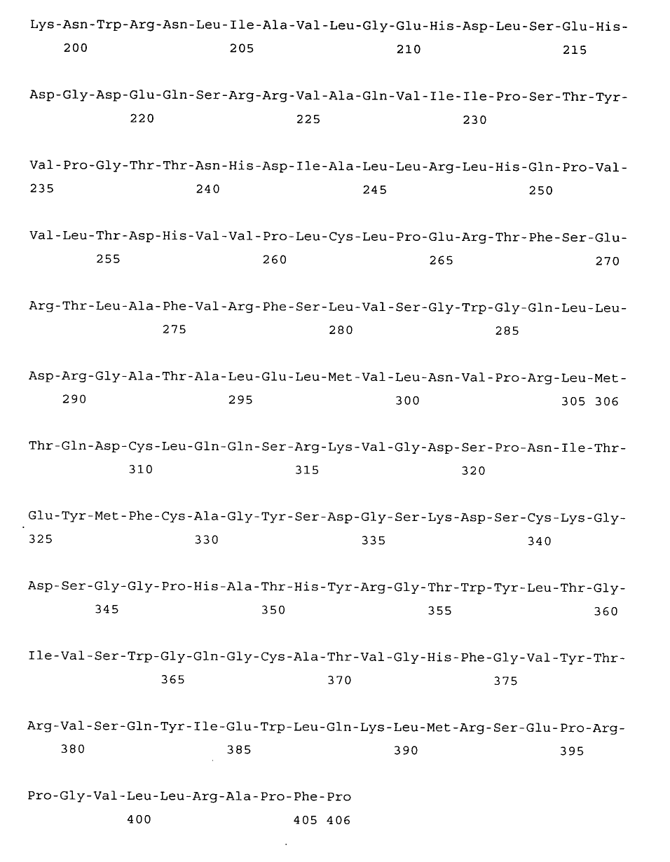

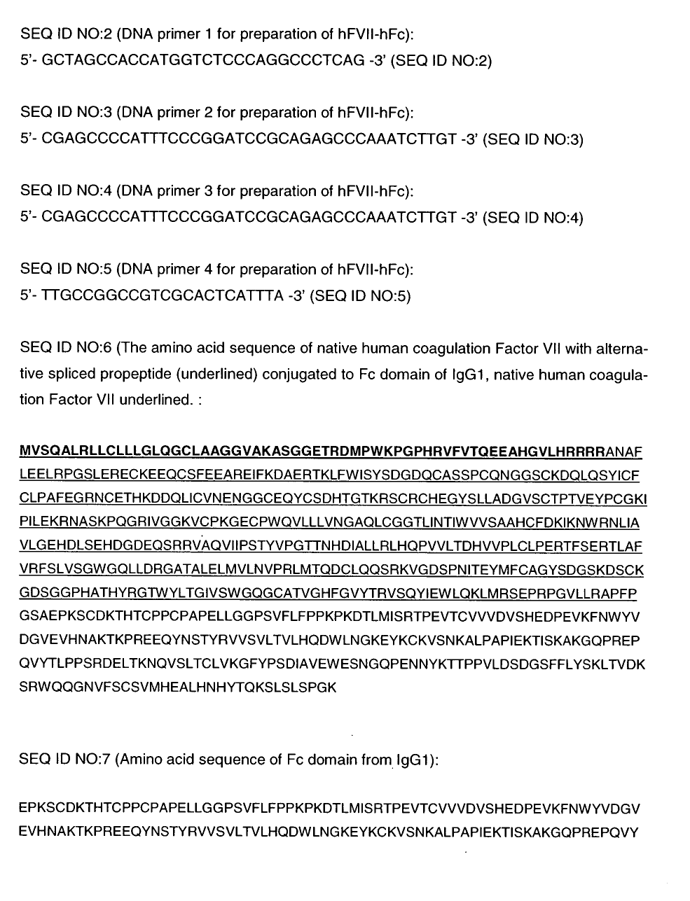

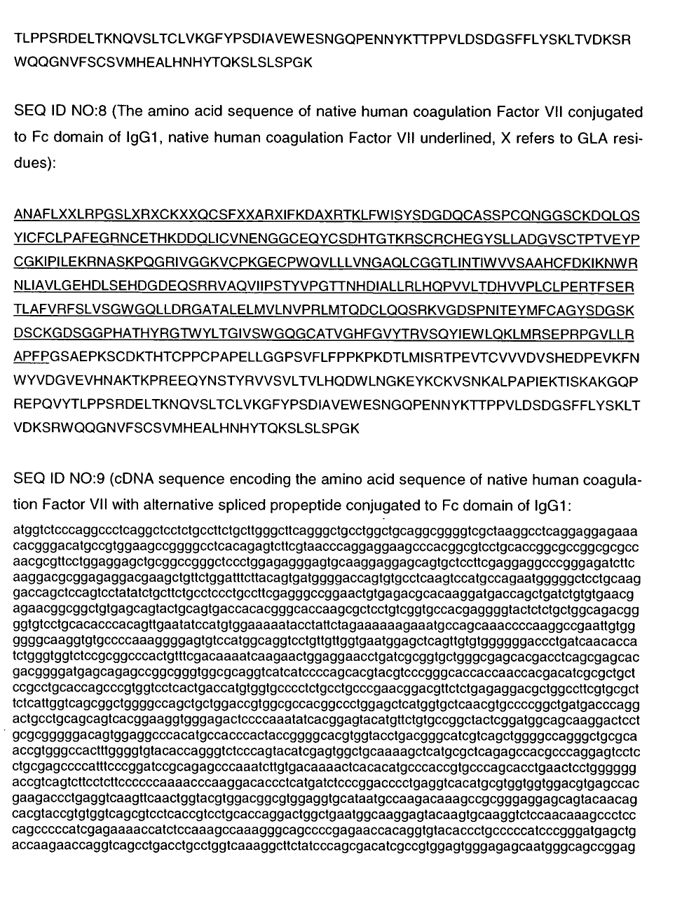

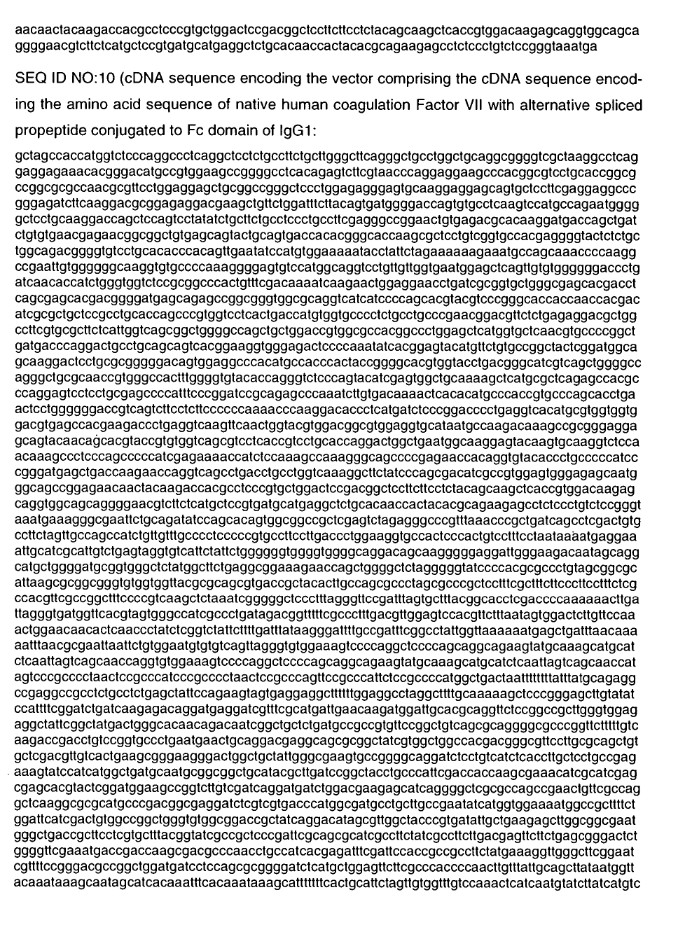

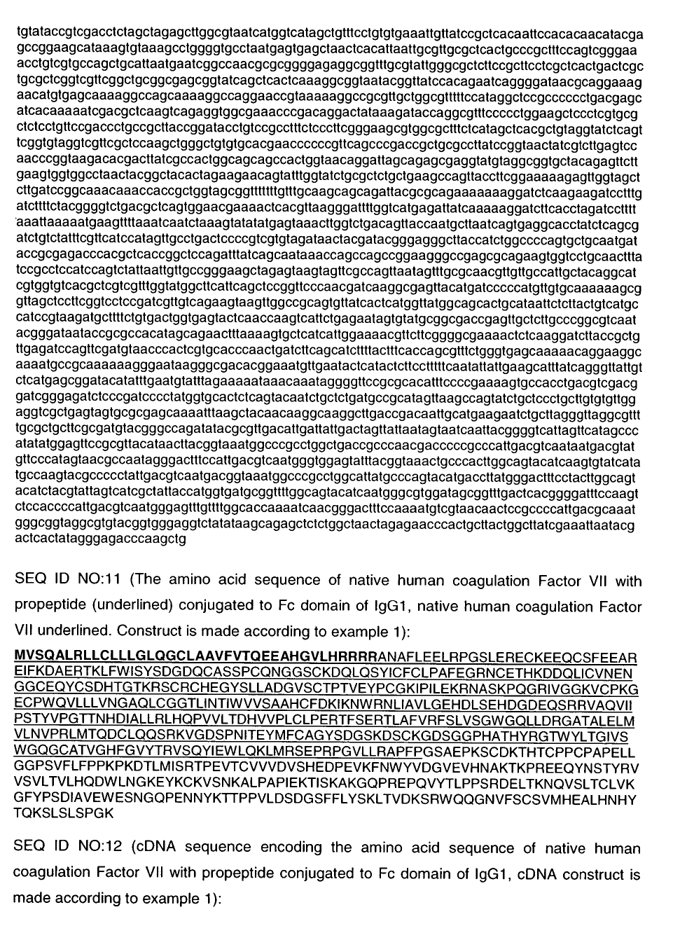

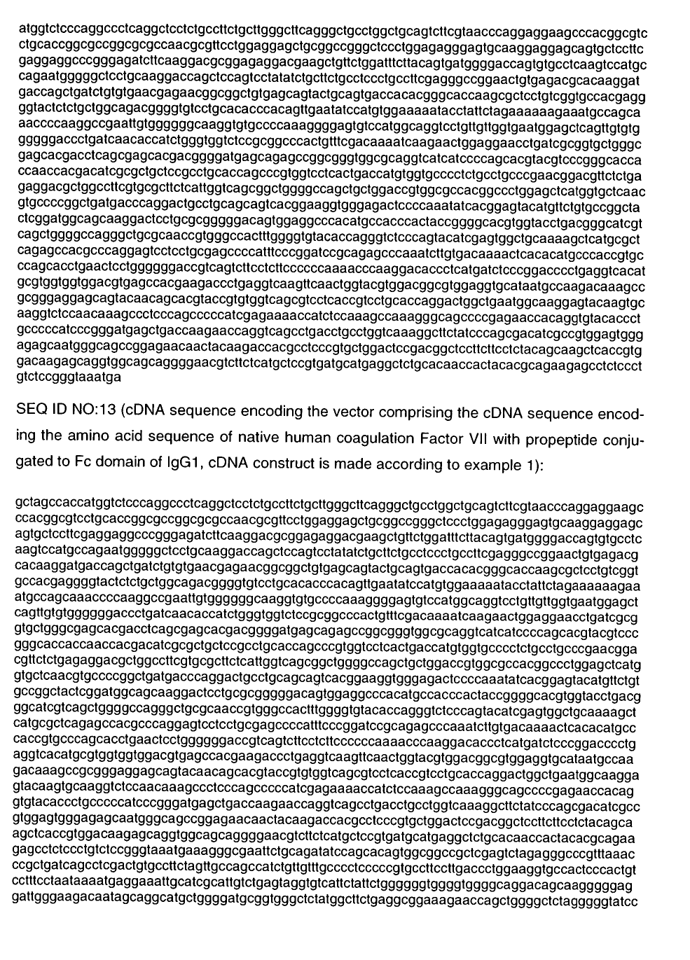

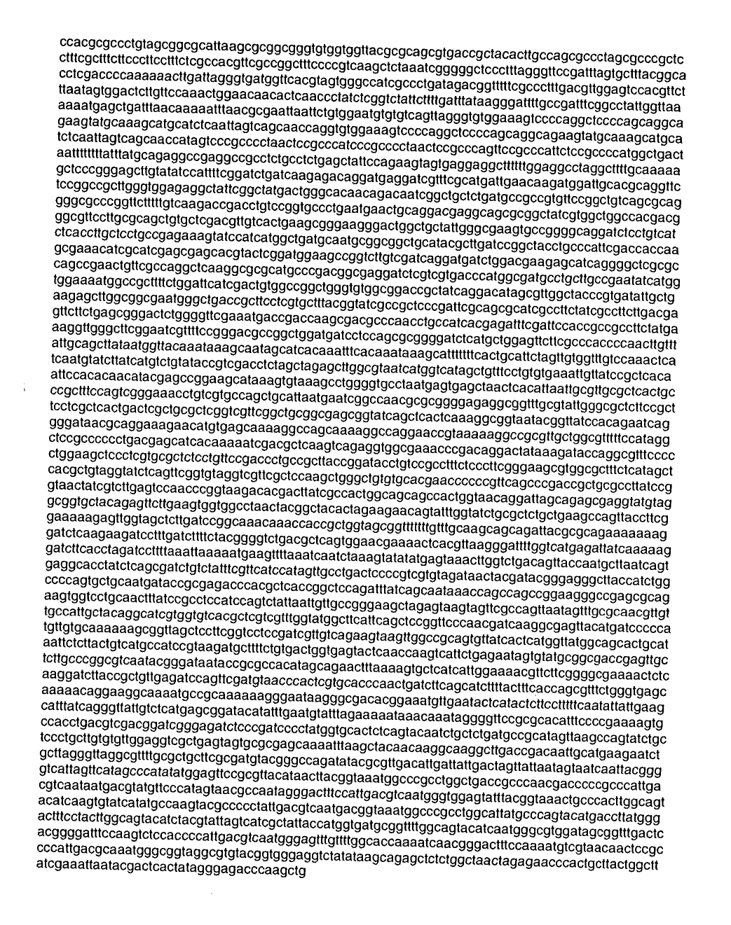

### DETAILED DESCRIPTION OF THE INVENTION

The present invention relates to conjugates of FVIIa polypeptides and an effector domain that act to elicit a cytolytic immune response. In one embodiment, the FVIIa polypeptides are chemically inactivated factor FVIIa polypeptides. An inactivated conjugate binds TF with high affinity and specificity but does not initiate blood coagulation. In one embodiment, the present invention relates to chemically inactivated immunoconjugates of factor FVIIa and the Fc region of a human immunoglobulin including the hinge region. The inactivated immunoconjugates bind TF with high affinity and specificity but do not initiate blood coagulation.

Currently no TF antagonists have been developed and marketed for therapeutic use in humans. Known therapeutic strategies include monoclonal antibodies, catalytically impaired FVIIa mutants, and chemical inactivated FVIIa. Native FVIIa binds TF with high affinity and most mutants with amino acid substitutions and monoclonal antibodies are expected to bind with equal or less affinity. The low binding affinity for TF may limit their effective use in the clinic. Chemical inactivated FVIIa has been reported to have a modest increase in the affinity for TF as compared to native FVIIa.

The reported inactive mutants of FVIIa and also the chemically inactivated FVIIa are expected to have short half lives comparable to that of circulating native FVII, i.e. 2-3 hours, which may limit the effective use in the clinic.

The present invention further relates to FVIIa polypeptides or active site inhibited derivatives thereof that are complexed or chemically coupled to a non-inhibitory anti-FVII antibody, i.e. an antibody, which do not block the FVII/TF complex formation. In one embodiment the antibody is an IgG sub-class antibody or fragments thereof. The antibody may subsequently be chemically coupled to FVII to form a stable, irreversible covalent complex.

The invention includes the following non-limiting derivatives:

1) The Fc domain of an antibody covalently coupled to FVIIa via an active site inhibitor. Here the FFR-cmk moiety may be chemically coupled to an Fc-domain generated either genetically or by proteolytic digestion of a selected antibody.

2) A non-inhibitory anti-FVII antibody bound non-covalently to FVII or FVIIai to form a complex with one or two FVII molecules and potentially elicit an immune response.

3) A non-inhibitory anti-FVII antibody chemically coupled to FVII or FVIIai to form a complex with one or two FVII molecules and potentially elicit an immune response.

4) FVII light chain (Gla to EGF1 or EGF2) chemically coupled to an antibody or Fc domain to form a complex with one or two FVII light chain molecules and potentially elicit an immune response.

5) An antibody directed against a non-natural C-terminal epitope genetically added to FVII. Same basic strategy as 2) and 3) but the antibody is directed against an exogenous epitope on FVII.

6) An antibody directed against an epitope added to FFR-cmk or similar active site inhibitor. Same basic strategy as 2) and 3) but the antibody is directed against an exogenous epitope on the active-site inhibitor.

While full length FVII does represent the preferred embodiment for 1, 2 and 3, the invention also encompasses the use of FVII (des-Gla) or any other TF-binding FVII derived protein, including truncated forms, analogs, derivatives and fusion proteins. The different affinity of such molecules for TF can provide a method for reducing the potentially undesirable effect of said compound on general haemostasis.

To increase the therapeutic potential of FVII, FVII analogs or chemical inactivated FVII, the present invention uses mutated or active site inhibited FVII or FVII analogs in complex with, or chemically coupled to, an anti-FVII antibody or Fc-domain of any IgG sub-type, depending on the desired immune response to the complex. This strategy allows for simple preparation of FVII:anti-FVII or FVII:Fc fusions which otherwise are complicated to produce as genetically engineered constructs due to the large complexity and requirement for post-translational processing. Furthermore, this strategy takes advantage of the fact that the FVII/TF complex is extremely tight (with binding affinity in the pM range) and the potential increase in binding affinity upon phosphatidylserine exposure. This same phenomenon is also the reason that it may prove difficult to produce monoclonal antibody with the required affinity for TF to efficiently compete with endogenous FVII. Furthermore, using the present invention allows the combination of the binding properties of FVII with the increased avidity resulting from the dimerization and the consequent ability to elicit a cytolytic response.

The increased avidity of the dimeric compound also enables the use of FVII light chain chemically coupled to a Fc-domain. In this embodiment, the efficacy would not as much depend on the competitive inhibition of FVII recruitment as on the potential for eliciting a cytolytic response.

Non-limiting examples of Factor VII polypeptides which may be used in the present invention having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993).

It is to be understood that the compound having the formula A-(LM)-C, wherein A is a FVIIa polypeptide; LM is an optional linker moiety; and C comprises an immunostimulatory effector domain, refers to compounds, wherein A, (LM), and C are chemically bound in the same entity. The chemical bonds may be covalent bonds, intermolecular hydrogen bonds, salt-bridge bonds, or other electrostatic interactions. In one embodiment, A, (LM), and C in the compound having the formula A-(LM)-C, wherein A is a FVIIa polypeptide; LM is an optional linker moiety; and C comprises an immunostimulatory effector domain, is bound together by covalent bonds.

The term “hybridoma cell” as used herein refers to cells produced by fusion of different cell types. Immunoglobulin molecules are normally synthesized by lymphoid cells derived from B lymphocytes of bone marrow. Lymphocytes can not be directly cultured over long periods of time to produce substantial amounts of their specific antibody. However, Kohler et al., 1975, Nature, 256:495, demonstrated that a process of somatic cell fusion, specifically between a lymphocyte and a myeloma cell, could yield hybridoma cells which grow in culture and produce a specific antibody called a “monoclonal antibody”. Myeloma cells are lymphocyte tumor cells which, depending upon the cell strain, frequently produce an antibody themselves, although “non-producing” strains are known.

In one embodiment of the invention, the compound having the formula A-(LM)-C inhibits TF-mediated FVIIa activity.

In one embodiment of the invention, A in the compound having the formula A-(LM)-C, is a FVIIa polypeptide that is catalytically inactivated in the active site.

In one embodiment of the invention, the compound having the formula A-(LM)-C inhibits TF-mediated coagulation activity.

In one embodiment of the invention, the compound having the formula A-(LM)-C inhibits TF-mediated signaling activity.

In one embodiment of the invention, the compound having the formula A-(LM)-C inhibits TF-mediated MAPK signaling.

In one embodiment of the invention, the compound having the formula A-(LM)-C inhibits the FVIIa-induced activation of the MAPK signaling.

In one embodiment of the invention, LM is present in the compound having the formula A-(LM)-C. In one embodiment of the invention, LM is absent in the compound having the formula A-(LM)-C. In one embodiment of the invention, C is an immunostimulatory effector domain.

In one embodiment of the invention, the disease or disorder associated with pathophysiological TF activity includes, without limitation, one or more of deep venous thrombosis, arterial thrombosis, post surgical thrombosis, coronary artery bypass graft (CABG), percutaneous transdermal coronary angioplastry (PTCA), stroke, cancer, tumor metastasis, angiogenesis, ischemia/reperfusion, rheumatoid arthritis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis.

The terms “FVIIa polypeptide” or “FVIIa polypeptides” as used herein means native Factor VIIa, as well as proteolytic functional equivalents of Factor VIIa that contain one or more amino acid sequence alterations relative to native Factor VIIa (i.e., Factor VII variants), and/or contain truncated amino acid sequences relative to native Factor VIIa (i.e., Factor VIIa fragments). Such equivalents may exhibit different properties relative to native Factor Vila, including stability, phospholipid binding, altered specific proteolytic activity, and the like.

As used herein, “Factor VII equivalent” encompasses, without limitation, equivalents of Factor VIIa exhibiting substantially the same or improved procoagulant activity relative to wild-type human Factor VIIa.

The terms “Factor VII” or “FVII” are intended to mean Factor VII polypeptides in their uncleaved (zymogen) form.

The terms “Factor VIIa” or “FVIIa” are intended to mean native bioactive forms of FVII. Typically, FVII is cleaved between residues 152 and 153 to yield FVIIa. The term “Factor VIIa” is also intended to encompass, without limitation, polypeptides having the amino acid sequence 1-406 of wild-type human Factor Vila (SEQ ID NO:1, as disclosed in U.S. Pat. No. 4,784,950), as well as wild-type Factor VIIa derived from other species, such as, e.g., bovine, porcine, canine, murine, and salmon Factor Vila. It further encompasses natural allelic variations of Factor VIIa that may exist and occur from one individual to another. Also, degree and location of glycosylation or other post-translation modifications may vary depending on the chosen host cells and the nature of the host cellular environment.

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. In one embodiment of the invention, the variant has a total amont of amino acid substitutions and/or additions and/or deletions independently selected from the group consisting of 1, 2, 3, 4, 5, 6, 7, 8, 9, and 10.

The term “active site” and the like when used herein with reference to FVIIa refers to the catalytic and zymogen substrate binding site, including the “S₁” site of FVIIa as that term is defined by Schecter, I. and Berger, A., (1967) Biochem. Biophys. Res. Commun. 7:157-162.

The term “TF-mediated FVIIa activity” as used herein means any TF-dependent activity. The term is intended to include both a TF-mediated coagulation activity and a signaling activity mediated by TF, e.g. MAPK signaling. In one embodiment, the TF-mediated FVIIa activity is MAPK signaling.

The term “TF-mediated MAPK signaling” is intended to mean events related to a cascade of intracellular events that mediate activation of Mitogen-Activated-Protein-Kinase (MAPK) or homologues thereof in response to the binding of a FVII polypeptide to TF. Three distinct groups of MAP kinases have been identified in mammalian cells: 1) extracellular-regulated kinase (Erk1/2 or p44/42), 2) c-Jun N-terminal kinase (JNK) and 3) p38 kinase. The Erk 1/2 pathway involves phosphorylation of Erk 1 (p 44) and/or Erk 2 (p 42). Activated MAP kinases e.g. p44/42 MAPK can translocate to the nucleus where they can phosphorylate and activate transcription factors including (Elk 1) and signal transducers and activators of transcription (Stat). Erk1/2 can also phosphorylate the kinase p90RSK in the cytoplasm or in the nucleus, and p90RSK then can activate several transcription factors. MAPK signaling may be measured as described in assay 6.

The term “protein kinase” is intended to indicate an enzyme that is capable of phosphorylating serine and/or threonine and/or tyrosine in peptides and/or proteins.

The term “FVIIa-induced activation of the MAPK signaling” is intended to indicate that FVIIa binds to TF in a mammalian cell and thereby induce MAPK signaling.

The term “TF-mediated coagulation activity” means coagulation initiated by TF through the formation of the TF/FVIIa complex and its activation of FIX and Factor X to FIXa and FXa, respectively. TF-mediated coagulation activity is measured in a FXa generation assay. The term “FXa generation assay” as used herein is intended to mean any assay where activation of FX is measured in a sample comprising TF, FVIIa, FX, calcium and phospholipids. An example of a FXa generation assay is described in assay 1.

By “catalytically inactivated in the active site” is meant that a FVIIa inhibitor is bound to the FVIIa polypeptide and decreases or prevents the FVIIa-catalysed conversion of FX to FXa. A FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The term “immunostimulatory effector domain” as used herein means a domain that is capable of stimulating an immune response in a mammal. Polypeptides appropriate for use as immunostimulatory effector domains include without limitation: opsonins such as IgG and C3b; proteins with carbohydrate residues that interact with the mannose-fucose receptor of phagocytes; proteins capable of recognition by receptors on scavenger macrophages; ligands for integrins; located on phagocytes; glycoproteins, such as integrins and selectins; and fucosyl transferase, which generates a Gal-Gal epitope recognized by macrophages.

The polypeptides of the immunostimulatory effector domain can be used in either a full-length or a truncated form, as appropriate. In particular, the terms “region” and “domain” as used to describe an immunostimulatory effector domain polypeptide includes either a full-length immunostimulatory effector domain polypeptide or a part of the immunostimulatory effector domain polypeptide, such as the IgG regions and domains described below.

Immunoglobulin G (IgG) is the preferred immunostimulatory effector domain polypeptide for use in this invention. An IgG protein contains (1) an Fab region (including the VH, VL and CH.sub.1 domains); (2) a hinge region, and (3) an Fc region (including the CH2 and CH3 domains). The Fab region is the region of an antibody protein which includes the antigen-binding portions. The “hinge” region is a flexible area on the immunoglobulin polypeptide that contains many residues of the amino acid proline and is where the Fc fragment joins one of the two Fab fragments. The Fc region, which is the constant region on an immunoglobulin polypeptide, is located on the immunoglobulin heavy chains and is not involved in binding antigens. The Fc region can bind to an Fc receptor on phagocytes. The amino-proximal end of the CH2 domain, especially amino acids 234 to 237, is important for binding of the Fc region to the Fc receptor. Fc receptors, such as Fc RI, are integral membrane proteins located on phagocytic white blood cells, such as macrophages. The hinge region is important for regulating Fc-Fc receptor interactions, providing flexibility to the polypeptide and functioning as a spacer.

The immunoglobulin polynucleotide used for producing a recombinant polypeptide immunostimulatory effector domain can be from any vertebrate, including, without limitation, human or mouse. Preferably, the polynucleotide encodes an immunoglobulin having a substantial number of sequences that are of the same origin as the host. For example, if a human is treated with a polypeptide of the invention, preferably the immunoglobulin is of human origin. The immunoglobulin polynucleotide may code for a full length polypeptide or a fragment, such as a fragment of a larger fusion protein, which includes an immunostimulatory polypeptide effector domain. Some advantages of using an immunoglobulin fusion protein include one or more of (1) possible increased avidity for multivalent ligands, (2) longer serum half-life, (3) the ability to activate effector cells by the Fc domain, and (4) ease of purification (for example, by protein A chromatography). Example 1 shows the construction of an immunoglobulin fusion protein according to the invention.

In one embodiment, IgG1 Fc is expressed on the cell surface in a “reverse orientation”. The Fc is in a reverse orientation (i.e. with the N-terminus projecting toward the FVIIa polypeptide and the C-terminus projecting away from the FVIIa polypeptide). Immunostimulatory Fc domains expressed in the reverse orientation retain the biological function of IgG1 Fc of binding Fc receptor to mediate macrophage activation, while simultaneously losing the complement fixation capability.

Polypeptides of the immunostimulatory effector domain and their receptors are important for the clearance and destruction of foreign materials, including mammalian cells or bacteria. Immunostimulatory cell surface polypeptides and their receptors activate the phagocytosis and ADCC. The process begins with opsonization of the foreign materials. An opsonin is an agent, usually an antibody or complement components, that makes a cell or microbe more vulnerable to being engulfed by a phagocyte; opsonization is the process of coating a cell with opsonin. A phagocyte is a cell that engulfs and devours another; the process of engulfing and devouring is phagocytosis. Among the important phagocytes are macrophages and monocytes. Monocytes are a type of large white blood cell that travels in the blood but which can leave the bloodstream and enter tissue to differentiate into macrophages. Macrophages digest debris and foreign cells. Monocytes are generally characterized by the cell surface expression of CD14.

In one embodiment of the invention, a FVIIa polypeptide conjugated with immunoglobulins binds phagocytes through the Fc receptors on the phagocytes. Phagocytes respond to signals from the Fc receptors by assembling cytoskeletal proteins, signaling cytoskeletal-protein assembly by activation of protein tyrosine kinases, and by phagocytosing the cell coated with immunoglobulin. IgG-Fc RI interaction activates various biological functions such as phagocytosis, endocytosis, ADCC, release of inflammatory mediators and superoxide anion production. Macrophages possess organic anion transporter proteins that promote the afflux of anionic substances from the macrophage. Thus, Fc RI mediates ADCC by macrophages and triggers both phagocytosis and superoxide production. For that reason, the compounds and methods of the invention, where the Fc domain of IgG is expressed on the FVIIa polypeptides to interact with phagocyte Fc receptor cause phagocytes to bind to the cell expressing TF, inducing ADCC. The IgG1 and IgG3 isotypes, that interact with the high affinity receptor Fc RI on macrophages, are preferred for the compounds and methods of the invention.

The complement-mediated cytotoxic activity (CMC activity) and antibody-dependent cell-mediated cytotoxicity (ADCC activity) of the FVIIa polypeptides conjugated with an effector domain may be measured by the methods of Ohta et al. [Cancer Immunol. Immunother., 36, 260 (1993)].

As stated above, the biological activity of antibodies is known to be determined, to a large extent, by the Fc region of the antibody molecule (Uananue and Benacerraf, Textbook of Immunology, 2nd Edition, Williams & Wilkins, p. 218 (1984)). This includes their ability to activate complement and to mediate ADCC as effected by leukocytes. Antibodies of different classes and subclasses differ in this respect, and, according to the present invention, antibodies of those classes having the desired biological activity are selected. For example, mouse immunoglobulins of the IgG3 and IgG2a class are capable of activating serum complement upon binding to the target cells.

In general, antibodies of the IgG1, IgG2a and IgG3 subclass can mediate ADCC, and antibodies of the IgG3, and IgG2a and IgM subclasses bind and activate serum complement. Complement activation generally requires the binding of at least two IgG molecules in close proximity on the target cell. However, the binding of only one IgM molecule activates serum complement.

The ability of any particular FVIIa polypeptide effector domain conjugate to mediate lysis of the tumor cell target by complement activation and/or ADCC can be assayed. The tumor cells of interest are grown and labeled in vivo; the FVIIa polypeptide effector domain conjugate is added to the tumor cell culture in combination with either serum complement or immune cells which may be activated by the antigen antibody complexes. Cytolysis of the target tumor cells is detected by the release of label from the lysed cells. In fact, FVIIa polypeptide effector domain conjugates can be screened using the patient's own serum as a source of complement and/or immune cells. The FVIIa polypeptide effector domain conjugate that is capable of activating complement or mediating ADCC in the in vitro test can then be used therapeutically in that particular patient.

In one embodiment of the invention, C in the compound having the formula A-(LM)-C comprises a molecule selected from the group consisting of mannose binding protein (MBP); proteins with carbohydrate residues that interact with the mannose-fucose receptor of phagocytes; opsonins such as IgG and C3b; proteins capable of recognition by receptors on scavenger macrophages; ligands for integrins normally located on phagocytes; glycoproteins, such as integrins and selectins; fucosyl transferase, which generates a Gal-Gal epitope recognized by macrophages.

In one embodiment of the invention, C in the compound having the formula A-(LM)-C is a molecule selected from the group consisting of mannose binding protein (MBP); proteins with carbohydrate residues that interact with the mannose-fucose receptor of phagocytes; opsonins such as IgG and C3b; proteins capable of recognition by receptors on scavenger macrophages; ligands for integrins normally located on phagocytes; glycoproteins, such as integrins and selectins; fucosyl transferase, which generates a Gal-Gal epitope recognized by macrophages.

In one embodiment of the invention, C in the compound having the formula A-(LM)-C comprises an immunoglobulin molecule or fragment thereof.

In one embodiment of the invention, C in the compound having the formula A-(LM)-C comprises an immunoglobulin molecule.

In one embodiment of the invention, C in the compound having the formula A-(LM)-C comprises an Fc domain or fragment thereof of an immunoglobulin molecule.

In one embodiment of the invention, C in the compound having the formula A-(LM)-C is an Fc domain or fragment thereof of an immunoglobulin molecule.

In one embodiment, the immunoglobulin molecule is selected from the group consisting of IgG1, IgG2, IgG3, IgM, IgA, IgE and IgD. In one embodiment, the immunoglobulin molecule is IgG. In one embodiment the immunoglobulin molecule is IgG1. In one embodiment, the immunoglobulin molecule is IgG2. In one embodiment, the immunoglobulin molecule is IgG3. In one embodiment, the immunoglobulin molecule is IgG4. In one embodiment, the immunoglobulin molecule is IgM. In one embodiment the immunoglobulin molecule is IgA. In one embodiment, the immunoglobulin molecule is IgE. In one embodiment the immunoglobulin molecule is IgD. In one embodiment, the immunoglobulin molecule is fully human. In one embodiment, the immunoglobulin molecule is a anti-FVII antibody. In one embodiment the immunoglobulin molecule is fully human. In one embodiment, the immunoglobulin molecule is a fully human anti-FVII antibody. In one embodiment, the anti-FVII antibody is a non-inhibitory antibody, which does not inhibit FVII/TF complex formation.

In one embodiment of the invention, C in the compound having the formula A-(LM)-C comprises the sequence of SEQ ID NO:7.

In one embodiment of the invention, C in the compound having the formula A-(LM)-C has the sequence of SEQ ID NO:7.

In one embodiment of the invention, the compound with the formula A-(LM)-C comprises the sequence of SEQ ID NO:8.

In one embodiment of the invention, the compound with the formula A-(LM)-C has the sequence of SEQ ID NO:8.

In one embodiment of the invention, C or (LM)-C in the compound having the formula A-(LM)-C is conjugated at the glycosylation side chains of the FVIIa polypeptide.

In one embodiment of the invention, C or (LM)-C in the compound having the formula A-(LM)-C is conjugated to a free sulfhydryl group present on the FVIIa polypeptide.

In one embodiment, the compound of the invention comprises more than one binding site for TF. In one embodiment, the compound is a dimer. In one embodiment, the compound is a trimer. In one embodiment, the compound is a tetramer. In one embodiment, the compound is a pentamer. In one embodiment, the compound is a hexamer.

In one embodiment of the invention, A in the compound having the formula A-(LM)-C is native human FVIIa or a fragment thereof.

In one embodiment of the invention, A in the compound having the formula A-(LM)-C is native human FVIIa.

In one embodiment of the invention, A in the compound having the formula A-(LM)-C is human FVIIa or a fragment thereof, which further comprises replacement of one, two, three, four or five amino acids in the N-terminal Gla domain (amino acids at position corresponding to 1-37 of SEQ ID NO:1) of Factor VIIa. This can provide the FVIIa polypeptide with a substantially higher affinity for membrane phospholipids, such as membrane phospholipids of tissue factor-bearing cells.

In one embodiment of the invention, A in the compound having the formula A-(LM)-C is native human FVIIa.

In one embodiment of the invention, the compound having the formula A-(LM)-C is not an immunoconjugate comprising the Fc region of a human IgG1 immunoglobulin and a mutant FVII polypeptide, that binds to TF but do not initiate blood clotting.

In one embodiment of the invention, LM in the compound having the formula A-(LM)-C comprises an amino acid sequence.

In one embodiment of the invention, LM in the compound having the formula A-(LM)-C is an amino acid sequence.

In one embodiment of the invention, LM in the compound having the formula A-(LM)-C comprises an amino acid sequence (Gly-Gly-Gly-Gly-Ser)ₙ, wherein n is any integer from 1 to 10.

In one embodiment of the invention, LM in the compound having the formula A-(LM)-C comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

In one embodiment of the invention, LM in the compound having the formula A-(LM)-C is a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

In one embodiment of the invention, A in the compound having the formula A-(LM)-C is catalytically inactivated in the active site with a chloromethyl ketone inhibitor independently selected from the group consisting of Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, Dansyl-D-Glu-Gly-Arg chloromethyl ketone.

The term “FFR-cmk” as used herein refers to D-Phe-Phe-Arg chloromethyl ketone.

The term “FFR-FVIIa” as used herein refers to FVIIa with a D-Phe-Phe-Arg chloromethyl ketone in the active site.

In one embodiment of the invention, LM in the compound having the formula A-(LM)-C comprises a chloromethyl ketone inhibitor independently selected from the group consisting of Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, Dansyl-D-Glu-Gly-Arg chloromethyl ketone, wherein A is catalytically inactivated in the active site with said chloromethyl ketone inhibitor. In one embodiment of the invention, LM in the compound having the formula A-(LM)-C is a chloromethyl ketone inhibitor independently selected from the group consisting of Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, Dansyl-D-Glu-Gly-Arg chloromethyl ketone, wherein A is catalytically inactivated in the active site with said chloromethyl ketone inhibitor.

Abbreviations Used Throughout the Description Include:

TF tissue factor

FVIIa activated factor VII

FXa factor Xa, activated factor X

FVII zymogen (single-strand, non-activated) factor VII

FX zymogen (single-strand, non-activated) factor X

TF/FVIa complex between TF and FVIIa

TF/FVIIa/FXa complex formed by FVIIa, TF and FXa

A TF/FVIIa mediated or associated process or event, or a process or event associated with TF-mediated coagulation activity, is any event that requires the presence of TF/FVIIa.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The no-reflow phenomenon, that is, lack of uniform perfusion to the microvasculature of a previously ischemic tissue has been described for the first time by Krug et al., (Circ. Res. 1966; 19:57-62).

The general mechanism of blood clot formation is reviewed by Ganong, in Review of Medical Physiology, 13^{th} ed., Lange, Los Altos Calif., pp 411-414 (1987). Coagulation requires the confluence of two processes, the production of thrombin which induces platelet aggregation and the formation of fribrin which renders the platelet plug stable. The process comprises several stages each requiring the presence of discrete proenzymes and profactors. The process ends in fibrin crosslinking and thrombus formation. Fibrinogen is converted to fibrin by the action of thrombin. Thrombin, in turn, is formed by the proteolytic cleavage of prothrombin. This proteolysis is effected by FXa which binds to the surface of activated platelets and in the presence of FVa and calcium, cleaves prothrombin. TF/FVIIa is required for the proteolytic activation of FX by the extrinsic pathway of coagulation. Therefore, a process mediated by or associated with TF/FVIIa, or a TF-mediated coagulation activity includes any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of FX to FXa, FIX to FIXa, and additional FVII to FVIIa. TF/FVIIa mediated or associated process, or TF-mediated coagulation activity can be conveniently measured employing standard assays such as those described in Roy, S., (1991) J. Biol. Chem. 266:4665-4668, and O'Brien, D. et al., (1988) J. Clin. Invest. 82:206-212 for the conversion of FX to FXa in the presence of TF/FVIIa and other necessary reagents.

The term “disease or disorder associated with pathophysiological TF activity” as used herein means any disease or disorder in which TF is involved. This includes, but is not limited to, diseases or disorders related to TF-mediated coagulation activity, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, cancer, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The disease or disorder associated with pathophysiological TF function is not limited to in vivo coagulopatic disorders such as those named above, but includes ex vivo TF/FVIIa related processes such as coagulation that may result from the extracorporeal circulation of blood, including blood removed in-line from a patient in such processes as dialysis procedures, blood filtration, or blood bypass during surgery.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

The terms “cancer or “tumor” are to be understood as referring to all forms of neo-plastic cell growth, including tumors of the lung, liver, blood cells (leukaemias), skin, pancreas, colon, prostate, uterus or breast.

It should be noted that peptides, proteins and amino acids as used herein can comprise or refer to “natural”, i.e., naturally occurring amino acids as well as “non.classical” D-amino acids including, but not limited to, the D-isomers of the common amino acids, α-isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, and amino acid analogues in general. In addition, the amino acids can include Abu, 2-amino butyric acid; γ-Abu, 4-aminobutyric acid; ε-Ahx, 6-aminohexanoic acid; Aib, 2-amino-isobutyric acid; β-Ala, 3-aminopropionic acid; Orn, ornithine; Hyp, trans-hydroxyproline; Nle, norleucine; Nva, norvaline.

The three-letter indication “GLA” as used herein means 4-carboxyglutamic acid (γ-carboxyglutamate).

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethyl ketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenyl-sulphonates and related compounds; heterocyclic protease inhibitors such as isocoumarines, and coumarins.

Examples of peptidic irreversible FVIIa inhibitors include, but are not limited to, Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, Dansyl-D-Glu-Gly-Arg chloromethyl ketone.

Examples of FVIIa inhibitors also include benzoxazinones or heterocyclic analogues thereof such as described in PCT/DK99/00138.

Examples of other FVIIa inhibitors include, but are not limited to, small peptides, peptidomimetics; benzamidine systems; heterocyclic structures substituted with one or more amidino groups; aromatic or heteroaromatic systems substituted with one or more C(═NH)NHR groups in which R is H, C₁₋₃alkyl, OH or a group which is easily split of in vivo.

By “linker moiety” or LM is meant any biocompatible molecule that links the effector domain to the FVIIa polypeptides. The FVIIa polypeptide and the effector domain are linked to the molecular LM via a chemical bond, e.g. via an amide or peptide bond between an amino group of the LM and a carboxyl group, or its equivalent, of the FVIIa polypeptide and the effector domain, or vice versa. It is to be understood, that the LM may contain both covalent and non-covalent chemical bonds or mixtures thereof. By “flexible” is meant that the LM comprises a plurality of carbon-carbon σ bonds having free rotation about their axes, so as to allow the FVIIa polypeptides and the effector domain to be separated by a distance suitable to both bind the TF site and elicit the effect of the effector domain.

Suitable LMs, or backbones, comprise, but are not limited to, group(s) such as peptides; polynucleotides; sacharides including monosaccharides, di- and oligosaccharides, cyclodextrins and dextran; polymers including polyethylene glycol, polypropylene glycol, polyvinyl alcohol, hydrocarbons, polyacrylates and amino-, hydroxy-, thio- or carboxy-functionalised silicones, other biocompatible material units; and combinations thereof. Such LM materials described above are widely commercially available or obtainable via synthetic organic methods commonly known to those skilled in the art.

The LM may, for example, be selected among the following structures:

straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl. The LM may be straight chained or branched and may contain one or more double or triple bonds. The LM may contain one or more heteroatoms like N, O or S. It is to be understood, that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

By “combinations thereof” is meant that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

The LM can comprise functional groups, such as, for example hydroxy, oxo, amino, C₁₋₄ monoalkylamino, acylamino, sulfonyl, carboxy, carboxamido, halogeno, C₁₋₆ alkoxy, C₁₋₆ alkylthio, trifluoroalkoxy, alkoxycarbonyl, or haloalkyl groups. The LM can also comprise charged functional groups, such as for example, ammonium groups or carboxylate groups.

The charged functional groups can provide TF antagonists with sufficient solubility in aqueous or physiological systems, provide reactive sites for ionic bonding with other species, and enhance their avidity to other members of the TF/FVIIa/FXa complex. It is within the purview of one of skill in the art to select a particular acid, and concentration thereof, to confer optimal solubility and avidity properties to the TF antagonists. Preferably, the total amount of charged functional groups are minimised so as to maximise the TF antagonists specificity for TF sites, but not so as to significantly decrease solubility.

The terms “C₁₋₅₀-alkyl” or “C₁₋₅₀-alkanediyl” as used herein, refer to a straight or branched, saturated or unsaturated hydrocarbon chain having from one to 50 carbon atoms.

The terms “C₂₋₅₀-alkenyl” or “C₂₋₅₀-alkenediyl” as used herein, refer to an unsaturated branched or straight hydrocarbon chain having from 2 to 50 carbon atoms and at least one double bond.

The terms “C₂₋₅₀-alkynyl” or “C₂₋₅₀-alkynediyl” as used herein, refer to an unsaturated branched or straight hydrocarbon chain having from 2 to 50 carbon atoms and at least one triple bond. The C₁₋₅₀-alkyl residues include aliphatic hydrocarbon residues, unsaturated aliphatic hydrocarbon residues, alicyclic hydrocarbon residues. Examples of a C₁₋₅₀-alkyl within this definition include but are not limited to decanyl, hexadecanyl, octadecanyl, nonadecanyl, icosanyl, docosanyl, tetracosanyl, triacontanyl, decanediyl, hexadecanediyl, octadecanediyl, nonadecanediyl, icosanediyl, docosanediyl, tetracosanediyl, triacontanediyl,

The term C₃₋₈-cycloalkyl means an alicyclic hydrocarbon residue including saturated alicyclic hydrocarbon residues having 3 to 8 carbon atoms such as cyclopropyl, cyclobutyl, cyclopentyl, cyclohexyl; and C₅₋₆ unsaturated alicyclic hydrocarbon residues having 5 to 6 carbon atoms such as 1-cyclopentenyl, 2-cyclopentenyl, 3-cyclopentenyl, 1-cyclohexenyl, 2-cyclohexenyl, 3-cyclohexenyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The term “C₁₋₆-alkylthio” as used herein, alone or in combination, refers to a straight or branched monovalent substituent comprising a C₁₋₆-alkyl group linked through an thioether sulfur atom having its free valence bond from the thioether sulfur and having 1 to 6 carbon atoms.

The terms “aryl” and “heteroaryl” as used herein refer to an aryl which can be optionally substituted or a heteroaryl which can be optionally substituted and includes phenyl, biphenyl, indene, fluorene, naphthyl(1-naphthyl, 2-naphthyl), anthracene(1-anthracenyl, 2-anthracenyl, 3-anthracenyl), thiophene(2-thienyl, 3-thienyl), furyl(2-furyl, 3-furyl), indolyl, oxadiazolyl, isoxazolyl, quinazolin, fluorenyl, xanthenyl, isoindanyl, benzhydryl, acridinyl, thiazolyl, pyrrolyl(2-pyrrolyl), pyrazolyl(3-pyrazolyl), imidazolyl(1-imidazolyl, 2-imidazolyl, 4-imidazolyl, 5-imidazolyl), triazolyl(1,2,3-triazol-1-yl, 1,2,3-triazol-2-yl 1,2,3-triazol-4-yl, 1,2,4-triazol-3-yl), oxazolyl(2-oxazolyl, 4-oxazolyl, 5-oxazolyl), thiazolyl(2-thiazolyl, 4-thiazolyl, 5-thiazolyl), pyridyl(2-pyridyl, 3-pyridyl, 4-pyridyl), pyrimidinyl(2-pyrimidinyl, 4-pyrimidinyl, 5-pyrimidinyl, 6-pyrimidinyl), pyrazinyl, pyridazinyl(3-pyridazinyl, 4-pyridazinyl, 5-pyridazinyl), quinolyl(2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl(2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl(2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl(1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl(1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl(1-benzoxazolyl, 2-benzoxazolyl), benzothiazolyl(1-benzothiazolyl, 2-benzothiazolyl, 4-benzothiazolyl, 5-benzothiazolyl, 6-benzothiazolyl, 7-benzothiazolyl), carbazolyl(1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The invention also relates to partly or fully saturated analogues of the ring systems mentioned above.

The terms “C₁₋₄ monoalkylamino” and “C₁₋₄ dialkylamino” refer to an amino group having one or both of its hydrogens independently replaced by an alkyl group having 1 to 4 carbon atoms, alkyl being defined above, such as methylamino, dimethylamino, N-ethyl-N-methylamino, ethylamino, diethylamino, propylamino, dipropylamino, N-(n-butyl)-N-methylamino, n-butylamino, di(n-butyl)amino, sec-butylammino, t-butylamino, and the like.

The terms “acyl” or “carboxy” refer to a monovalent substituent comprising a C₁₋₆-alkyl group linked through a carbonyl group; such as e.g. acetyl, propionyl, butyryl, isobutyryl, pivaloyl, valeryl, and the like.

The term “acylamino” refers to the group C₁₋ₙ C(═O)NH—

The term “carboxamido” refers to the group —C(═O)NHC₁₋ₙ

The term “trifluoroalkoxy” refers to an C₁₋₆ alkoxy group as defined above having three of its hydrogen atoms bonded to one or more of the carbon atoms replaced by fluor atoms, such as (CF₃)O—, (CF₃)CH₂O—.

The term “alkoxycarbonyl” refers to the group —C(═O)(R) where R is an C₁₋₆ alkoxy group as defined above. The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “leaving group” as used herein includes, but is not limited to, halogen, sulfonate or an acyl group. Suitable leaving groups will be known to a person skilled in the art.

“Halogen” refers to fluorine, chlorine, bromine, and iodine. “Halo” refers to fluoro, chloro, bromo and iodo.

“Optional” or “optionally” means that the subsequently described event or circumstances may or may not occur, and that the description includes instances where said event or circumstance occur and instances in which is does not. For example, “aryl . . . optionally substituted” means that the aryl may or may not be substituted and that the description includes both unsubstituted aryls and aryls wherein there is substitution

The immunostimulatory effector domain conjugates C-(LM) comprising a FVIIa inhibitor to be used in the preparation of a TF antagonist may be prepared by the following methods. In the following methods the FVIIa inhibitor is designated the letter F. The immunostimulatory effector domain C is designated the letter C. Linker part B refers to other linker part of the LM.

Method 1.

LM comprising FVIIa inhibitors is prepared by reacting F-B-X, in which X is a functional group capable of reacting with structures C-Y, in which Y is a functional group, by means of normal coupling reactions using coupling reagents known by the person skilled in the art.

Method 2.

LM comprising FVIIa inhibitors may be prepared by reaction between F-B-Z, in which Z is a leaving group and C-W in which W is a nucleophile. Examples of leaving groups are halogens, sulfonates, phosphonates. Examples of nucleophiles are hydroxy, amino, N-substituted amino, and carbanions.

Method 3.

LM comprising FVIIa inhibitors may be prepared by reaction between C-B-Z, in which Z is a leaving group, and F-W, in which W is a nucleophile. Examples of leaving groups are halogens, sulfonates, phosphonates. Examples of nucleophiles are hydroxy amino, N-substituted amino, and carbanions.

Method 4.

The linker part B can be reacted with structures F and C connected to a solid phase surface using methods well known in the art.

Method 5.

The immunostimulatory effector domain conjugates C-(LM) comprising a FVIIa inhibitor may be prepared by a sequence of reactions through which F or C firstly are reacted with the activated linker moiety forming F-B, respectively C-B moieties and subsequently the formed product is reacted with C, respectively F moiety. The actual bond formation taking place through reaction on functional groups or derivatives or leaving groups/nucleophiles as described under methods 1-3.

The reaction can be carried out in solution phase or on a solid phase support using the procedures known by the person skilled in the art.

In the present specification, amino acids are represented using abbreviations, as indicated in table 1, approved by IUPAC-IUB Commission on Biochemical Nomenclature (CBN). Amino acid and the like having isomers represented by name or the following abbreviations are in natural L-form unless otherwise indicated. Further, the left and right ends of an amino acid sequence of a peptide are, respectively, the N- and C-termini unless otherwise specified.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The invention also relates to a method of preparing human FVIIa polypeptides as mentioned above. The human FVIIa polypeptides are preferably produced by recombinant DNA techniques. To this end, DNA sequences encoding human FVIIa may be isolated by preparing a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the protein is preferably of human origin, i.e. derived from a human genomic DNA or cDNA library.

The DNA sequences encoding the human FVIIa polypeptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the human FVIIa polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human FVIIa polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVIIa polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the human FVIIa polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVIIa sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVIIa polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human FVIIa polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human FVIIa polypeptides into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human FVIIa polypeptides. The function of the leader peptide is to allow the expressed peptide to be directed from the endoplasmic reticulum to the Golgi apparatus and further to a secretory vesicle for secretion into the culture medium (i.e. exportation of the human FVIIa polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the human FVIIa polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the human FVIIa polypeptides of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVIIa polypeptide of interest.

The host cell into which the DNA sequences encoding the human FVIIa polypeptides is introduced may be any cell, which is capable of producing the posttranslational modified human FVIIa polypeptides and includes yeast, fungi and higher eukaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220,1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVIIa polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the human FVIIa polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The human FVIIa polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

For the preparation of recombinant human FVIIa polypeptides, a cloned wild-type FVIIa DNA sequence is used. This sequence may be modified to encode a desired FVIIa variant. The complete nucleotide and amino acid sequences for human FVIIa are known. See U.S. Pat. No. 4,784,950, which is incorporated herein by reference, where the cloning and expression of recombinant human FVIIa is described. The bovine FVIIa sequence is described in Takeya et al., J. Biol. Chem, 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVIIa protein to obtain proper post-translational processing (e.g. gamma-carboxylation of glutamic acid residues) and secretion from the host cell. The pre-pro peptide may be that of FVIIa or another vitamin K-dependent plasma protein, such as factor IX, factor X, prothrombin, protein C or protein S. As will be appreciated by those skilled in the art, additional modifications can be made in the amino acid sequence of FVIIa where those modifications do not significantly impair the ability of the protein to act as a coagulation factor. For example, FVIIa in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the human FVIIa polypeptide. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the sequence encoding the human FVIIa polypeptide is replaced with corresponding sequences from a milk specific protein gene. Such replacement provides a putative tissue-specific initiation environment to enhance expression. It is convenient to replace the entire pre-pro sequence of the human FVIIa polypeptide and 5′ non-coding sequences with those of, for example, the BLG gene, although smaller regions may be replaced.

For expression of a human FVIIa polypeptide in transgenic animals, a DNA segment encoding the human FVIIa polypeptide is operably linked to additional DNA segments required for its expression to produce expression units. Such additional segments include the above-mentioned promoter, as well as sequences which provide for termination of transcription and polyadenylation of mRNA. The expression units will further include a DNA segment encoding a secretory signal sequence operably linked to the segment encoding the human FVIIa polypeptide. The secretory signal sequence may be a native secretory signal sequence of the human FVIIa polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the human FVIIa polypeptide into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVIIa polypeptide, thereby creating a gene fusion that includes the expression control sequences of the milk protein gene. In any event, cloning of the expression units in plasmids or other vectors facilitates the amplification of the human FVIIa polypeptide. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49: 113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); and Brinster et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Patent Office Publication EP 255,378.

FVIIa produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol. Chem, 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVIIa described herein (see, generally, Scopes, R., Protein Purification, Springer-Verlag, N.Y., 1982). Substantially pure FVIIa of at least about 90 to 95% homogeneity is preferred, and 98 to 99% or more homogeneity most preferred, for pharmaceutical uses. Once purified, partially or to homogeneity as desired, the FVIIa may then be used therapeutically.

Conversion of single-chain FVII to active two-chain FVIIa may be achieved using factor XIIa as described by Hedner and Kisiel (1983, J. Clin. Invest. 71: 1836-1841), or with other proteases having trypsin-like specificity (Kisiel and Fujikawa, Behring Inst. Mitt. 73: 29-42, 1983). Alternatively FVII may be autoactivated by passing it through an ion-exchange chromatography column, such as mono Q.RTM. (Pharmacia Fire Chemicals) or the like (Bjoern et al., 1986, Research Disclosures 269:564-565). The FVIIa molecules of the present invention and pharmaceutical compositions thereof are particularly useful for administration to humans to treat a variety of conditions involving intravascular coagulation.

The compounds of the present invention may have one or more asymmetric centres and it is intended that stereoisomers (optical isomers), as separated, pure or partially purified stereoisomers or racemic mixtures thereof are included in the scope of the invention.

Within the present invention, the TF antagonist may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) which are known to the skilled artisan.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

The TF antagonists of the invention are useful for the preparation of a pharmaceutical composition for the treatment of or prophylaxis of thrombotic or coagulopathic related diseases or disorders including vascular diseases and inflammatory responses. Such diseases and responses include, but are not limited to deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis.

The TF antagonist may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Apart from the pharmaceutical use of the compounds, they may be useful in vitro tools for investigating the inhibition of FVIIa, FXa or TF/FVIIa/FXa activity.

Pharmaceutical Compositions

In another aspect, the present invention includes within its scope pharmaceutical compositions comprising a TF antagonist, as an active ingredient, or a pharmaceutically acceptable salt thereof together with a pharmaceutically acceptable carrier or diluent.

Optionally, the pharmaceutical composition of the invention may comprise a TF antagonist in combination with one or more other compounds exhibiting anticoagulant activity, including, without limitation, a platelet aggregation inhibitor.

The compounds of the invention may be formulated into pharmaceutical composition comprising the compounds and a pharmaceutically acceptable carrier or diluent. Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

The compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

| | | |
|---|---|---|
| Core: | | |
| Active compound (as free compound | 10 | mg |
| or salt thereof) | | |
| Colloidal silicon dioxide (Areosil ®) | 1.5 | mg |
| Cellulose, microcryst. (Avicel ®) | 70 | mg |
| Modified cellulose gum (Ac-Di-Sol ®) | 7.5 | mg |
| Magnesium stearate | | |
| Coating: | | |
| HPMC | approx. 9 | mg |
| *Mywacett ® 9-40 T | approx. 0.9 | mg |

*Acylated monoglyceride used as plasticizer for film coating.

The compounds of the invention may be administered to a mammal, especially a human in need of such treatment, prevention, elimination, alleviation or amelioration of various thrombolytic or coagulophatic diseases or disorders as mentioned above. Such mammals also include animals, both domestic animals, e.g. household pets, and non-domestic animals such as wildlife.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

The compounds of the present invention have interesting pharmacological properties. For example, the compounds of this invention can be used to modulate and normalise an impaired haemostatic balance in mammals caused by deficiency or malfunction of blood clotting factors or their inhibitors. The FVIIa and in particular the TF/FVIIa activity plays an important role in the control of the coagulation cascade, and modulators of this key regulatory activity such as the present invention can be used in the treatment of or prophylaxis of thrombotic or coagulopathic related diseases or disorders including vascular diseases and inflammatory responses. The pharmaceutical composition of the invention may thus be useful for modulating and normalising an impaired haemostatic balance in a mammal. In particular, the pharmaceutical composition may be useful for the treatment of or prophylaxis of thrombotic or coagulopathic related diseases or disorders including vascular diseases and inflammatory responses.

“Modulating and normalising an impaired haemostatic balance” means achieving an effect on the coagulation system measurable in vitro assays and/or animal models which diminishes the risk for thrombosis or bleedings.

More particularly, the pharmaceutical composition may be useful as an inhibitor of blood coagulation in a mammal, as an inhibitor of clotting activity in a mammal, as an inhibitor of deposition of fibrin in a mammal, as an inhibitor of platelet deposition in a mammal, in the treatment of mammals suffering from deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis. The compositions of the invention may also be used as an adjunct in thrombolytic therapy.

Furthermore the invention relates to a method for inhibiting the TF initiation activity in a mammal which method comprises administering an effective amount of at least one compound of the present invention, in combination with a pharmaceutical acceptable excipient and/or carrier to the mammal in need of such a treatment.

Assays

Inhibition of FVIIa/Phospholipids-Embedded TF-Catalyzed Activation of FX by TF Antagonists FXa Generation Assay (Assay 1):

In the following example all concentrations are final. Lipidated TF (10 pM), FVIIa (100 pM) and TF antagonist or FFR-rFVIIa (0-50 nM) in HBS/BSA (50 mM hepes, pH 7.4, 150 mM NaCl, 5 mM CaCl₂, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for TF antagonist inhibition of FVIIa/lipidated TF-mediated activation of FX may be calculated. The IC₅₀ value for FFR-rFVIIa is 51+/−26 pM in this assay.

Inhibition of FVIIa/Cell Surface TF-Catalyzed Activation of FX by TF Antagonists (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102KerTr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with with 1 mg/ml BSA and 5 mM Ca²⁺). FVIIa (1 nM), FX (135 nM) and varying concentrations of TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC50 value for FFR-rFVIIa is 1.5 nM in this assay.

Inhibition of ¹²⁵I-FVIIa Binding to Cell Surface TF by TF Antagonists (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (10 mM Hepes, pH 7.45, 150 mM NaCl, 4 mM KCl, and 11 mM glucose) supplemented with 5 mM EDTA and then once with buffer A and once with buffer B (buffer A supplemented with with 1 mg/ml BSA and 5 mM Ca²⁺). The monolayers are preincubated 2 min with 100 μl cold buffer B. Varying concentrations of Mabs (or FFR-FVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC50 value for FFR-rFVIIa is 4 nM in this assay.

Assay to Measure the Ability of a FVIIa Polypeptide Effector Domain Conjugate to Mediate Lysis of the Tumor Cell Target by Complement-Mediated Cytolysis (CMC) and/or ADCC (Assay 4):

The assay is performed essentially as described in Hudson and Hay: Practical Immunology 2^{nd} edition 1980, p279 with mouse lymphocytes and rabbit anti-(mouse) lymphocyte antiserum and guinea pig complement. Briefly, the complement is absorbed with a tumor cell line expressing human TF, e.g. J82 (ATCC number HTB-1), alternatively a CHO cell line transfected with full length human TF; approximately 0.1 ml packed cells per ml serum, for 30 min at 4° C. The absorbed complement is centrifuged and stored at −20° C.

The tumor cells expressing human TF are labeled with ⁵¹Cr, and washed. A dilution series of the FVIIa polypeptide effector domain conjugate, e.g. FVIIa-Fc polypeptide is mixed with 0.1 ml complement (To measure ADCC freshly isolated peripheral blood leukocytes at a ratio of 60 leukocytes per tumor cell is added) and 0.1 ml of 5×10⁶ labeled cells per ml, and incubated at 37° C. for 30 min. The final volume is adjusted to 0.5 ml, and after mixing and centrifugation (150 g for 10 min at 4° C.), the released isotope in 0.1 ml supernatant is measured. The amount of ⁵¹Cr released into the medium is used to calculate the % cytolysis as compared to a control.

Biosensor Assay (Assay 5):

TF antagonists are tested on the Biacore instrument by passing a standard solution of the TF antagonist over a chip with immobilized TF. This is followed by different concentrations of sTF in 10 mM hepes pH 7.4 containing 150 mM NaCl, 10 mM CaCl₂ and 0.0003% polysorbate 20. Kd's are calculated from the sensorgrams using the integrated Biacore evaluation software.

Inhibition of FVIIa/TF-Induced p44/42 MAPK Activation by TF Antagonists with Effector Domain (Assay 6):

The amount of phosphorylated p44/42 MAPK and/or Akt, and/or p90RSK is determined by quantitative detection of chemiluminescence (Fujifilm LAS-1000) from western blot analysis. Cells expressing human TF, e.g. CCD1102KerTr, NHEK P166, human glioblastoma cell line U87, or human breast cancer cell line MDA-MB231, are cultured in medium with 0-0.1% FCS for 24 or 48 hours prior to the experiment to make cells quiescent. At the day of the experiment the cells must be 70-80% confluent. The experiment is performed by preincubating the cells with excess TF antagonist or FFR-rFVIIa in medium without serum for 30 min at 37° C. before addition of 10-100 nM FVIIa and incubating for 10 min. As a positive control of cell signaling, cells are treated with 10% FCS for 10 minutes. Cells are washed 2 times in ice-cold PBS before cells are lysed in lysis buffer (20 mM Tris, 0.1% Triton X-100, 1 mM EDTA, 1 mM EGTA, 50 mM sodium-fluoride, 10 mM sodium-glycerophosphate, 5 mM sodium pyrophosphate, 150 mM NaCl, pH 7.5 containing 0.1 mM 4-(2-aminoethyl)benzenesulfonyl fluoride (AEBSF) and 1 mM benzamidine. Added just before use: 1 mM sodium orthovanadate, 5 μg/ml leupeptin, 10 μg/ml aprotinin). Lysates were mixed with SDS-sample buffer and loaded on a SDS-polyacrylamide gel. A standard biotinylated protein marker is loaded on each gel. Proteins separated on the SDS-polyacrylamide gel were transferred to nitrocellulose by electroblotting, and the kinases p44/42 MAPK, Akt and p90RSK were visualized by immunoblotting with phosphospecific antibodies, and chemiluminiscence is quanitiated by Fujifilm LAS1000.

The present invention is further illustrated by the following examples.

The present invention is not to be limited in scope by the specific embodiments disclosed in the examples which are intended as illustrations of a number of aspects of the invention and any embodiments which are functionally equivalent are within the scope of this invention. Those skilled in the art will know, or be able to ascertain using no more than routine experimentation, many equivalents to the specific embodiments of the invention described herein. These and all other equivalents are intended to be encompassed by the following claims.

### EXAMPLES

### Example 1

Generation and Expression of hFVII-hFc.

A plasmid vector pFVII-Fc for expression of human FVII-human Fc(IgG1) fusion protein in mammalian cells is generated based on the pcDNA3.1+ plasmid vector (Invitrogen, Carlsbad, Calif.). Briefly, the pFVII-Fc vector carries the cDNA nucleotide sequence encoding human FVII including the propeptide, fused to the Fc fragment of human IgG1, under the control of a strong CMV promoter for transcription of the inserted cDNA, and neomycin phosphotransferase cDNA under the control of an SV40 early promoter for use as a selectable marker. A FVII cDNA insert is generated from a full-length FVII cDNA plasmid (pLN174, Persson and Nielsen, 1996, FEBS Letters, 385, 241-243), but any full length FVII cDNA clone from e.g. a human liver cDNA library could be used, by PCR using Expand High Fidelity (Roche). The PCR product is generated using the oligonucleotides Primer 1 and 2, by procedures for preparing a DNA construct using polymerase chain reaction using specific primers that are well known to persons skilled in the art (cf. PCR Protocols, 1990, Academic Press, San Diego, Calif., USA):

| | | |
|---|---|---|
| Primer 1: | 5′-GCTAGCCACCATGGTCTCCCAGGCCCTCAG-3′ | (SEQ ID NO:2) |
| Primer 2: | 5′-CGAGCCCCATTTCCCGGATCCGCAGAGCCCAAATCTTGT-3′ | (SEQ ID NO:3) |

This PCR reaction generates a FVII cDNA including an in-frame segment encoding Gly-Ser-Ala and the 5′ cDNA sequence of the Fc fragment of human IgG1. The Fc fragment of human IgG1 is amplified from a Human Lymph Node cDNA library (Clontech, BD Biosciences, Franklin Lakes, N.J., USA), but any cDNA library containing IgG1 could be used, using oligonucleotides Primer 3 and 4:

| | | |
|---|---|---|
| Primer 3: | 5′-CGAGCCCCATTTCCCGGATCCGCAGAGCCCAAATCTTGT-3′ | (SEQ ID NO:4) |
| Primer 4: | 5′-TTGCCGGCCGTCGCACTCATTTA-3′ | (SEQ ID NO:5) |

This PCR reaction generates a cDNA sequence of the Fc fragment with the 3′ terminal of human FVII cDNA and an in-frame segment encoding Gly-Ser-Ala, 5′ to the human Fc cDNA sequence. DNA from both PCR reactions are mixed and a new PCR reaction performed using Primer 1 and Primer 4, yielding a fusion protein containing the FVII cDNA, an in-frame segment encoding Gly-Ser-Ala, and a cDNA encoding the Fc fragment of human IgG1. The PCR product is cloned into pCR2.1-TOPO using a topoisomerase cloning kit as per manufacture's instructions (Invitrogen, Carlsbad, Calif.), resulting in an intermediate plasmid, pCR-FVII-Fc. The procedure for moving the complete hFVII-hFc cDNA to pcDNA3.1+ using NheI and EcoRI restriction enzymes is well known to persons skilled in the art (cf. Molecular Cloning, 2001, Cold Spring Habor Laboratory Press, Cold Spring Habor, N.Y., USA):

Following a restriction reaction with NheI and EcoRI restriction enzymes of the pCR-FVII-Fc plasmid, the DNA fragment containing the FVII-Fc cDNA is isolated using agarose gel electrophoresis. The purified insert is ligated into the NheI and EcoRI site of pcDNA3.1+ vector using T4 DNA ligase and transformed into an appropriate E. coli strain, e.g. DH5α or XL1 Blue; plasmid vectors containing the desired cDNA sequence are identified and isolated using standard techniques and the sequence is verified by DNA sequencing. The resulting expression vector, pFVII-Fc, will encode the following protein:

hFVII-GSA-hFc(IqG1)

The pFVII-Fc vector is transfected into CHO cells using Lipofectamine, or similar technique, and stable clones are isolated following neomycin selection. Clones secreting FVII-Fc are identified using a FVII ELISA and any high producing clones will be further subcloned to yield a clone with a high specific FVII-Fc expression in Dulbecco-modified Eagle's medium with 10% fetal calf serum. The clone will subsequently be adapted to serum free suspension culture using a commercially available CHO medium (JRH Bioscience). The resulting recombinant FVII-Fc material can then be purified from the media using conventional methods (Thim, L. et al. Biochemistry (1988) 27:7785-93).

### Example 2

Expression of FVII-Fc.

CHO cells or BHK cells are transfected with the pFVII-Fc vector essentially as previously described (Thim et al. (1988) Biochemistry 27, 7785-7793; Persson and Nielsen (1996) FEBS Lett. 385, 241-243) to obtain expression of FVII-Fc. The FVII-Fc protein is purified as follows: Conditioned medium is loaded onto a 25-ml column of Protein-G Sepharose (Pharmacia Biotech) equilibrated in 20 mM Tris, 100 mM NaCl, pH 7.4. The column is washed in the equilibration buffer, and elution of the protein is accomplished by 0.1 M glycine, pH 2.7. The pH in the fractions containing the FVII-Fc protein is adjusted to pH 7.5 by titration with 2 M Tris, and the pooled fractions are dialysed against 50 mM Hepes, pH 7.5, containing 10 mM CaCl₂, 100 mM NaCl and 0.02% Triton X-100, before the application to a 25-ml column containing monoclonal antibody F1A2 (Novo Nordisk, Bagsværd, Denmark) coupled to CNBr-activated Sepharose 4B (Pharmacia Biotech).

The column is equilibrated with 50 mM Hepes, pH 7.5, containing 10 mM CaCl₂, 100 mM NaCl and 0.02% Triton X-100. After washing with equilibration buffer and equilibration buffer containing 2 M NaCl, bound material is eluted with equilibration buffer containing 10 mM EDTA instead of CaCl₂. Before use or storage, excess CaCl₂ over EDTA is added or FVII-Fc is transferred to a Ca²⁺-containing buffer. The yield of each step was followed by factor VII ELISA measurements and the purified protein was analysed by SDS-PAGE. Subsequent inactivation of the FVIIa moiety of FVII-Fc molecules by e.g. FFR-cmk are known to the person skilled in the art.

### Example 3

Synthesis of FVIIa Polypeptides Covalently Conjugated to Anti-FVII Antibody.

1) Simple Chemical X-Linking:

a) Glutaraldehyde:

Catalytically active FVIIa or FVIIa inhibited with FFR-cmk are mixed with a stoichiometric amount of an anti-FVII antibody (1 antigen binding site per FVII molecule) which do not prevent TF binding is dialyzed against reaction buffer (50 mM HEPES, 100 mM NaCl, 10 mM CaCl₂, pH 7.5) to eliminate any free primary amines and the protein concentration is adjusted to 2.4 μM. The appropriate concentration of glutaraldehyde is determined by mixing equal volumes of the protein solution with aqueous glutaraldehyde solutions at different concentrations (500 mM to 32 μM) for 5 min at room temperature. The reaction is then quenched by addition of 10 mM NH₄OH. The extent of X-linking is then assessed by precipitation of an aliquot by adding ½ volume of 80% trichloroacetic acid and separation of the reaction products on a reducing SDS/PAGE or analytic HPLC. Once the appropriate concentration for a particular antibody FVII combination has been established the reaction is scaled up using this particular concentration of glutaraldehyde and the components separated by binding of the complex to an anion exchange matrix in the presence of 10 mM EDTA and elution of the bound protein by CaCl₂ to remove unbound anti-sera followed gelfiltration to separate complexes from free components.

b) Bifunctional X-Linkers:

Protocol is essentially a described above, except for the use of bifunctional X-linker (As described with examples in Pierce Catalogue).

2) Directed Chemical X-Linking:

a) Hetero-Bifunctional X-Linkers:

These linkers contains two distinct functionalities, typically they contain one reacting with primary amines and a thiol-reagent (e.g. malemide, pyridyl or iodo-) or a photo-activated group. Catalytically active FVIIa, FVIIai or an anti FVII antibody, which do not prevent TF binding, is suspended in dialyzed against reaction buffer (50 mM HEPES, 100 mM NaCl, 10 mM CaCl₂, pH 7.5) to eliminate any free primary amines, and the protein concentration is adjusted to 2.4 μM. When dealing with an amine specific reagent, this moiety has to be reacted before any other functionality. Furthermore, as a general rule for thiol-specific reagents FVII need to be treated before addition of the antibody, however, in the case of FVIIai in which the active site is blocked by a thiol containing inhibitor, e.g. Ac-Cys-D-Phe-Phe.Arg-cmk, and the use of photoactivated linkers the order of reaction is not important. The appropriate concentration of X-linker is determined by mixing equal volumes of the protein solution with aqueous X-linker solutions at 2-4 fold excess and incubated for 1-2 hours at room temperature. The reaction is then quenched by addition of 10 mM NH₄OH followed by gelfiltration or dialysis to remove excess reagent. The protein to be coupled is then added at stoichiometric amounts (i.e., 1 antigen binding site per FVII molecule). For thiol-reactive X-linkers the intact antibody is first partially reduced by TCEP before addition to pre-labelled FVIIa and the resulting mixture is incubated over night at 4° C., while for photoactivated linkers the mixture is by exposed to long wave UV light for 15 minutes at a distance of 3.5 cm at room temperature. The extent of X-linking is then assessed by precipitation of an aliquot by adding ½ volume of 80% trichloroacetic acid and separation of the reaction products on a reducing SDS/PAGE or by analytical HPLC. Once the appropriate concentration for a particular antibody FVII combination has been established the reaction is scaled up using this particular concentration of X-linker and the components separated by binding of the complex to an anion exchange matrix in the presence of 10 mM EDTA and elution of the bound protein by CaCl₂ to remove unbound anti-sera followed gelfiltration to separate complexes from free components. The final product is analyzed for is ability to bind TF using BiaCore and other suitable assays which are well described in the literature.

3) X-Linking Via Specifically Derivatized FFR-cmk:

a) Thiol Reactive Moiety:

The same basic scheme at described for thiol-specific reagents above, however, with the major exception that the thiol-reactive probe is introduced into FVIIa as part of a highly specific active site inhibitor. The inhibitor is introduced into the active site of FVII as previously described for production of FVIIai or ASIS.

b) Photoactivated Moiety:

The same basic scheme at described for thiol-specific reagents above, however, with the major exception that the photo-activated probe is introduced into FVIIa as part of a highly specific active site inhibitor. The inhibitor is introduced into the active site of FVII as previously described for production of FVIIai or ASIS.

1. A compound having the formula A-(LM)-C, wherein A is a Factor VIIa (FVIIa) polypeptide; LM is an optional linker moiety; and C comprises an immunostimulatory effector domain; and wherein said compound binds to tissue factor (TF).

2. The compound according to claim 1, wherein said compound inhibits TF-mediated FVIIa activity.

3. The compound according to claim 1, wherein A is a FVIIa polypeptide that is catalytically inactivated in the active site.

4. The compound according to claim 3, wherein A is catalytically inactivated in the active site with a chloromethyl ketone inhibitor independently selected from the group consisting of Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, and Dansyl-D-Glu-Gly-Arg chloromethyl ketone.

5. The compound according to claim 1, wherein A is native human FVIIa or a fragment thereof.

6. The compound according to claim 5, wherein A is native human FVIIa.

7. The compound according to claim 1, wherein C comprises a molecule selected from the group consisting of: mannose binding protein (MBP); proteins with carbohydrate residues that interact with the mannose-fucose receptor of phagocytes; opsonins; proteins capable of recognition by receptors on scavenger macrophages; ligands for integrins normally located on phagocytes; and glycoproteins comprising a Gal-Gal epitope recognized by macrophages.

8. The compound according to claim 1, wherein C comprises an immunoglobulin molecule or fragment thereof.

9. The compound according to claim 1, wherein C comprises an immunoglobulin molecule.

10. The compound according to claim 8, wherein C comprises an Fc domain of an immunoglobulin molecule or fragment thereof.

11. The compound according to claim 8, wherein the immunoglobulin molecule is selected from the group consisting of IgG1, IgG2, IgG3, IgM, IgA, IgE and IgD.

12. The compound according to claim 11, wherein the immunoglobulin molecule is selected from the group consisting of IgG1 and IgG3.

13. The compound according to claim 8, wherein the immunoglobulin molecule is fully human.

14. The compound according to claim 8, wherein the immunoglobulin molecule is an anti-FVII antibody.

15. The compound according to claim 14, wherein the anti-FVII antibody does not inhibit FVII/TF complex formation.

16. The compound according to claim 1, wherein C comprises the sequence of SEQ ID NO:7.

17. The compound according to claim 1, wherein the compound with the formula A-(LM)-C comprises the sequence of SEQ ID NO:8.

18. The compound according to claim 1, wherein C or (LM)-C is conjugated to oligosaccharides present on the FVIIa polypeptide.

19. The compound according to claim 1, wherein C or (LM)-C is conjugated to a free sulfhydryl group present on the FVIIa polypeptide.

20. The compound according to claim 1, wherein the compound comprises more than one binding site for TF.

21. The compound according to claim 1, wherein LM comprises an amino acid sequence.

22. The compound according to claim 21, wherein the LM comprises the amino acid sequence (Gly-Gly-Gly-Gly-Ser)ₙ, wherein n is any integer from 1 to 10.

23. The compound according to claim 1, wherein LM comprises a molecule selected from the group consisting of: straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, wherein the molecules are optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆alkylthio, trifluoroalkoxy, alkoxycarbonyl, haloalkyl.

24. The compound according to claim 1, wherein LM comprises a chloromethyl ketone inhibitor independently selected from the group consisting of Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, and Dansyl-D-Glu-Gly-Arg chloromethyl ketone, wherein A is catalytically inactivated in the active site with said chloromethyl ketone inhibitor.

25. A pharmaceutical composition comprising (i) an amount of a compound having the formula A-(LM)-C, wherein A is a FVIIa polypeptide; LM is an optional linker moiety; C comprises an immunostimulatory effector domain; and wherein said compound binds to TF; and (ii) a pharmaceutically acceptable carrier or excipient.

26. A compound for use as a medicament having the formula A-(LM)-C, wherein A is a FVIIa polypeptide; LM is an optional linker moiety; C comprises an immunostimulatory effector domain; and wherein said compound binds to TF.

27. A method for preventing or treating disease or disorder associated with pathophysiological TF activity, said method comprising contacting a TF expressing cell with a compound having the formula A-(LM)-C, wherein A is a FVIIa polypeptide; LM is an optional linker moiety; C comprises an immunostimulatory effector domain; and wherein said compound binds to TF.

28. A method according to claim 27, wherein said disease or disorder associated with pathophysiological TF activity is selected from the group consisting of: deep venous thrombosis, arterial thrombosis, post surgical thrombosis, coronary artery bypass graft (CABG), percutaneous transdermal coronary angioplastry (PTCA), stroke, cancer, tumor metastasis, angiogenesis, ischemia/reperfusion, rheumatoid arthritis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, wherein said method comprises administering a therapeutically effective amount of said compound in combination with a pharmaceutical acceptable excipient and/or carrier, to a mammal in need of such a treatment.
